(19) United States
(12) Patent Application Publication
(10) Pub. No.: US 2020/0078303 A1
(43) Pub. Date: Mar. 12, 2020

(54) PHARMACEUTICAL FORMULATIONS OF SUVOREXANT

(71) Applicant: Druggability Technologies IP HoldCo Ltd., Visida (MT)

(72) Inventors: Erzsébet Réka ANGI, Nagykovácsi (HU); Orsolya BASA-DÉNES, Eger (HU); Zsolt ÖTVÖS, Csongrád (HU); Hristos GLAVINAS, Szeged (HU); Genovéva FILIPCSEI, Budapest (HU)

(21) Appl. No.: 16/469,014

(22) PCT Filed: Dec. 19, 2017
(86) PCT No.: PCT/US2017/067328

§ 371 Date: Jun. 12, 2019

Related U.S. Application Data
(60) Provisional application No. 62/436,195, filed on Dec. 19, 2016.

(51) Int. Cl. A61K 9/16; A61K 31/5513; A61K 9/00; A61K 9/19

(52) U.S. Cl. CPC A61K 9/1617; A61K 31/5513; A61K 9/0053; B82Y 5/00; A61K 9/1641; A61K 9/1652; A61K 9/19; A61K 9/1635

(57) ABSTRACT

The present invention relates to pharmaceutical formulations comprising as active compound Suvorexant, or its salts, or its metabolites or derivatives, thereof and pharmaceutical excipients, process for the preparation thereof and pharmaceutical compositions containing them. The pharmaceutical formulations of the present invention possess instantaneous redispersibility, increased apparent solubility and permeability, no observable food effect with respect to immediate absorption and more predictable plasma concentration throughout the night and next morning. The invention also relates to methods of manufacturing the pharmaceutical formulations and pharmaceutical compositions containing them according to the invention, their uses and methods of treatments using the pharmaceutical formulations and their pharmaceutical compositions.

### FIELD OF THE INVENTION

The invention is directed to a stable pharmaceutical formulations with controlled particle size, increased apparent solubility and increased dissolution rate comprising as active compound Suvorexant, or its salts, or its metabolites or derivatives thereof, which is useful in the treatment of insomnia characterized by difficulties with sleep onset and/or sleep maintenance. More specifically, the pharmaceutical formulations of the present invention possess instantaneous redispersibility, increased apparent solubility and permeability, no observable food effect with respect to immediate absorption and more predictable plasma concentration throughout the night and next morning. The invention also relates to methods of manufacturing the pharmaceutical formulations and pharmaceutical compositions containing them according to the invention, their uses and methods of treatments using the pharmaceutical formulations and their pharmaceutical compositions.

### BACKGROUND OF THE INVENTION

Suvorexant is described chemically as: [(7R)-4-(5-chloro-2-benzoxazolyl) hexahydro-7-methyl-1H-1,4-diazepin-1-yl][5-methyl-2-(2H-1,2,3-triazol-2-yl)phenyl]methanone. Its empirical formula is C₂₃H₃ClN₆O₂ and the molecular weight is 450.92. Its structural formula is:

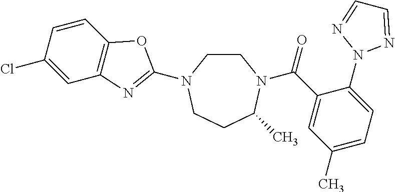 [SMILES: Cc1ccc(-n2nccn2)c(C(=O)N2CCN(c3nc4cc(Cl)ccc4o3)CC[C@H]2C)c1]

Suvorexant is a white to off-white powder that is insoluble in water.

BELSOMRA tablets contain Suvorexant, a highly selective antagonist for orexin receptors OX1R and OX2R.

Each film coated tablet contains 5 mg, 10 mg, 15 mg, or 20 mg of Suvorexant and the following inactive ingredients: polyvinylpyrrolidone/vinyl acetate copolymer (copovidone), microcrystalline cellulose, lactose monohydrate, croscarmellose sodium, and magnesium stearate.

In addition, the film coating contains the following inactive ingredients: lactose monohydrate, hypromellose, titanium dioxide, and triacetin. The film coating for the 5 mg tablets also contains iron oxide yellow and iron oxide black, and the film coating for the 10 mg tablets also contains iron oxide yellow and FD&C Blue #1/Brilliant Blue FCF Aluminum Lake.

The mechanism by which Suvorexant exerts its therapeutic effect in insomnia is presumed to be through antagonism of orexin receptors. The orexin neuropeptide signaling system is a central promoter of wakefulness. Blocking the binding of wake-promoting neuropeptides orexin A and orexin B to receptors OX1R and OX2R is thought to suppress wake drive. Antagonism of orexin receptors may also underlie potential adverse effects such as signs of narcolepsy/cataplexy. Genetic mutations in the orexin system in animals result in hereditary narcolepsy; loss of orexin neurons has been reported in humans with narcolepsy.

Suvorexant exposure increases in a less than strictly dose-proportional manner over the range of 10-80 mg because of decreased absorption at higher doses. Suvorexant pharmacokinetics are similar in healthy subjects and patients with insomnia.

Suvorexant peak concentrations occur at a median tₘₐₓ of 2 hours (range 30 minutes to 6 hours) under fasted conditions. The mean absolute bioavailability of 10 mg is 82%.

Ingestion of Suvorexant with a high-fat meal resulted in no meaningful change in AUC or Cₘₐₓ but a delay in tₘₐₓ of approximately 1.5 hours. Suvorexant may be taken with or without food; however for faster sleep onset, Suvorexant should not be administered with or soon after a meal.

The mean volume of distribution of Suvorexant is approximately 49 liters. Suvorexant is extensively bound (>99%) to human plasma proteins and does not preferentially distribute into red blood cells. Suvorexant binds to both human serum albumin and α1-acid glycoprotein.

Suvorexant is mainly eliminated by metabolism, primarily by CYP3A with a minor contribution from CYP2C19. The major circulating entities are Suvorexant and a hydroxy-suvorexant metabolite. This metabolite is not expected to be pharmacologically active.

The primary route of elimination is through the feces, with approximately 66% of radiolabeled dose recovered in the feces compared to 23% in the urine. The systemic pharmacokinetics of Suvorexant are linear with an accumulation of approximately 1- to 2-fold with once-daily dosing. Steady-state is achieved by 3 days. The mean t½ is approximately 12 hours (95% CI: 12 to 13).

The main pharmacokinetic problem associated with the oral delivery of Suvorexant is the unpredictable absorption profile which results in unpredictable onset of action, unpredictable plasma concentrations through the night and the next morning leading to next day drowsiness.

In order to overcome the problems associated with prior conventional Suvorexant formulations and available drug delivery systems, novel pharmaceutical formulations of Suvorexant or its salts, its metabolites or its derivatives thereof using pharmaceutical excipients were prepared. The novel pharmaceutical formulations of the present invention possess instantaneous redispersibility, increased apparent solubility and permeability, no observable food effect with respect to immediate absorption and more predictable plasma concentration throughout the night and next morning.

A variety of strategies have been used to attempt to overcome these issues, see for example Uditi Handa et al, World Journal of Pharmaceutical and Life Sciences, 2(3) (2016)171-188, Alfred C. F. Rumondor et al., Journal of Pharmaceutical Sciences 105 (2016) 2498-2508, Kesisoglou F et al., J Pharm Sci. 104(9) (2015) 2913-2922, WO/2015/158910, WO/2014/072961 and US201261653539.

### BRIEF DESCRIPTION OF THE INVENTION

1. Stable pharmaceutical formulations with improved physicochemical characteristics and enhanced biological performance comprising
   i. Suvorexant, or its salt, or its metabolite or derivatives thereof;
   ii. at least one primary pharmaceutical excipients chosen from poloxamers (copolymers of ethylene oxide and propylene oxide blocks), copolymers of vinylpyrrolidone and vinyl acetate copolymer, polyvinylpyrrolidone, polyvinyl caprolactam-polyvinyl acetate-polyethylene glycol graft copolymer, ethylene oxide/propylene oxide tetra functional block copolymer, hydroxypropylcellulose and d-alpha tocopheryl polyethylene glycol 1000 succinate; and
   iii. optionally, secondary pharmaceutical excipients;
      wherein said pharmaceutical formulations have particle size is between 10 nm and 600 nm, and possesses one or more among the following features:
   a) is instantaneously redispersible in physiological relevant media;
   b) is stable in solid form and in colloid solution and/or dispersion;
   c) has apparent solubility in water is of at least 0.15 mg/mL;
   d) has PAMPA permeability of at least 6·10⁻⁶ cm/s when dispersed in FaSSiF or FeSSiF media, which does not decrease in time at least for 2 months;
   e) exhibits no observable food effect with respect to immediate absorption and more predictable plasma concentration throughout the night and next morning.
2. The pharmaceutical formulations according to Point 1, wherein said pharmaceutical formulations have particle size in the range between 10 nm and 600 nm.
3. The pharmaceutical formulations according to Point 2, wherein said pharmaceutical formulations have particle size in the range between 10 nm and 400 nm.
4. The pharmaceutical formulations according to Point 1, wherein said pharmaceutical formulations exhibits X-ray amorphous character in the solid form.
5. The pharmaceutical formulations according to Point 1, wherein said pharmaceutical formulations possesses at least two of the properties described in a)-e).
6. The pharmaceutical formulations according to Point 5, wherein said pharmaceutical formulations possesses at least three of the properties described in a)-e).
7. The pharmaceutical formulations according to Point 6, wherein said pharmaceutical formulations possess instantaneous redispersibility, have apparent solubility in water of at least 0.15 mg/mL, exhibits no observable food with respect to immediate absorption and more predictable plasma concentration throughout the night and next morning. This could result in more predictable plasma concentrations lowering the incidence of adverse events leading to untimely dose reduction or discontinuation of the therapy.
8. The pharmaceutical formulations according to Point 6, wherein said pharmaceutical formulations possess instantaneous redispersibility, have PAMPA permeability of at least 6·10⁻⁶ cm/s when dispersed in FaSSIF or FeSSIF biorelevant media, which does not decrease in time at least for 2 month, exhibits no observable food effect with respect to immediate absorption and more predictable plasma concentration throughout the night and next morning.
9. The pharmaceutical formulations according to Point 5, wherein said pharmaceutical formulations have apparent solubility in water of at least 0.15 mg/mL and PAMPA permeability of at least 6·10⁻⁶ cm/s.
10. The pharmaceutical formulations according to Point 6, wherein said pharmaceutical formulations possess instantaneous redispersibility, have apparent solubility in water of at least 0.15 mg/mL, and have PAMPA permeability of at least 6·10⁻⁶ cm/s.
11. The pharmaceutical formulations according to Point 1, wherein said primary pharmaceutical excipient is polyvinyl caprolactam-polyvinyl acetate-polyethylene glycol graft copolymer or hydroxypropylcellulose.
12. The pharmaceutical formulations according to Point 1, wherein said secondary pharmaceutical excipient is chosen from cetylpyridinium chloride, poloxamers (copolymers of ethylene oxide and propylene oxide blocks), mannitol, polyglycol mono- and di-esters of 12-hydroxystearic acid, dioctyl sodium sulfosuccinate, sodium acetate and sodium lauryl sulfate.
13. The pharmaceutical formulations according to Point 13, wherein said secondary pharmaceutical excipient is dioctyl sodium sulfosuccinate, sodium lauryl sulfate and mannitol.
14. The pharmaceutical formulation 1 according to Point 1 comprising
   a) Suvorexant;
   b) polyvinyl caprolactam-polyvinyl acetate-polyethylene glycol graft copolymer as primary pharmaceutical excipient; and
   c) dioctyl sodium sulfosuccinate as secondary pharmaceutical excipient;
      wherein said pharmaceutical formulation has characteristic Raman shifts at 426, 568, 640, 680, 701, 794, 846, 880, 921, 953, 1037, 1087, 1199, 1249, 1335, 1374, 1402, 1448, 1505, 1571, 1591, 1616, 1635, 1736, 2691, 2860 and 2938 cm⁻¹; and ATR peaks at 571, 601, 717, 840, 951, 974, 1031, 1084, 1148, 1196, 1236, 1334, 1371, 1421, 1442, 1478, 1570, 1631, 1732, 2857 and 2926 cm
15. The pharmaceutical formulation 2 according to Point 1 comprising
   a) Suvorexant;
   b) hydroxypropylcellulose as primary pharmaceutical excipient; and
   c) sodium lauryl sulfate and mannitol as secondary pharmaceutical excipients;
      wherein said pharmaceutical formulation has characteristic Raman shifts at 474, 639, 845, 876, 887, 924, 953, 1053, 1084, 1112, 1129, 1146, 1250, 1297, 1376, 1404, 1453, 1508, 1572, 1587, 1615, 2728, 2850, 2882, 2937, 2918 and 2963 cm⁻¹; and ATR peaks at 592, 626, 716, 837, 892, 931, 1026, 1082, 1220, 1251, 1376, 1453, 1571, 1639, 2920, 2848 and 2964 cm⁻¹.
16. A pharmaceutical formulation according to either of Point 1 or Point 13 comprising a primary pharmaceutical excipient which is polyvinyl caprolactam-polyvinyl acetate-polyethylene glycol graft copolymer and secondary pharmaceutical excipient which is dioctyl sodium sulfosuccinate, in a total amount ranging from about 1.0 weight % to about 95.0 weight % based on the total weight of the pharmaceutical formulation.
17. A pharmaceutical formulation according to either of Point 1 or Point 13 comprising a primary pharmaceutical excipient which is polyvinyl caprolactam-polyvinyl acetate-polyethylene glycol graft copolymer and secondary pharmaceutical excipients which is dioctyl sodium sulfosuccinate, in a total amount ranging from about 50.0 weight % to about 95.0 weight % based on the total weight of the pharmaceutical formulation.
18. A pharmaceutical formulation according to either of Point 1 or Point 13 comprising a primary pharmaceutical excipient which is hydroxypropylcellulose and secondary pharmaceutical excipient which are sodium lauryl sulfate and mannitol, in a total amount ranging from about 1.0 weight % to about 95.0 weight % based on the total weight of the pharmaceutical formulation.
19. A pharmaceutical formulation according to either of Point 1 or Point 13 comprising a primary pharmaceutical excipient which is hydroxypropylcellulose and secondary pharmaceutical excipients which are sodium lauryl sulfate and mannitol, in a total amount ranging from about 50.0 weight % to about 95.0 weight % based on the total weight of the pharmaceutical formulation.
20. The pharmaceutical formulations according to Point 1, wherein said pharmaceutical formulations have increased dissolution rate.
21. A process for the preparation of stable pharmaceutical formulations according to Point 1, said process comprising the step of mixing a pharmaceutically acceptable solution of Suvorexant and at least one primary pharmaceutical excipient which is polyvinyl caprolactam-polyvinyl acetate-polyethylene glycol graft copolymer or hydroxypropylcellulose with an aqueous solution containing at least one secondary pharmaceutically accepted excipients which are dioctyl sodium sulfosuccinate; or sodium lauryl sulfate and mannitol.
22. The process according to Point 21, wherein said process is performed in a continuous flow instrument.
23. The process according to Point 22, wherein said continuous flow instrument is a microfluidic flow instrument.
24. The process according to Point 21, wherein the pharmaceutically acceptable solvent of said pharmaceutically acceptable solution is chosen from methanol, ethanol, isopropanol, n-propanol, acetone, acetonitrile, tetrahydrofuran, or combinations thereof.
25. The process according to Point 24, wherein the pharmaceutically acceptable solvent of said pharmaceutically acceptable solutions are isopropanol and n-propanol which are mixed with said aqueous solutions of Point 19.
26. The process according to Point 21, wherein said pharmaceutically acceptable solutions are miscible with said aqueous solution and the aqueous solution comprises 0.1 to 99.9% weight of the final solution.
27. A pharmaceutical composition comprising the stable pharmaceutical formulation according to Point 1 together with a pharmaceutically acceptable carriers.
28. The pharmaceutical composition according to Point 27, wherein said pharmaceutical composition is suitable for oral, pulmonary, rectal, colonic, parenteral, intracisternal, intravaginal, intraperitoneal, ocular, otic, local, buccal, nasal, or topical administration.
29. The pharmaceutical composition according to Point 28, wherein said composition is suitable for oral administration.
30. Pharmaceutical formulations according to Point 1 for use in the treatment of insomnia characterized by difficulties with sleep onset and/or sleep maintenance, in improving daytime sleep in shift workers, in the treatment of insomnia related to bipolar disorder, in the treatment of Suvorexant and trauma related insomnia, in the treatment of insomnia in Parkinson disease, in the treatment of sleep pressure in hypertensives with insomnia and in the treatment of insomnia in Alzheimer's disease.
31. A method of treatment of insomnia characterized by difficulties with sleep onset and/or sleep maintenance, improving daytime sleep in shift workers, treatment of insomnia related to bipolar disorder, treatment of Suvorexant and trauma related insomnia, treatment of insomnia in Parkinson disease, treatment of sleep pressure in hypertensives with insomnia and treatment of insomnia in Alzheimer's disease comprising administration of a therapeutically effective amount of the pharmaceutical formulation according to Point 1 or the pharmaceutical composition according to Point 29.
32. Stable pharmaceutical formulations comprising
   a) 5-40% by weight of Suvorexant, or its salt or its metabolites or derivatives thereof;
   b) 20-90% by weight of a polyvinyl caprolactam-polyvinyl acetate-polyethylene glycol graft copolymer or hydroxypropylcellulose;
   c) 5-40% by weight of dioctyl sodium sulfosuccinate or sodium lauryl sulfate and mannitol; and
      wherein said pharmaceutical formulations have controlled particle size in the range between 10 nm and 600 nm; and
      wherein said pharmaceutical formulations are not obtained via a milling process, high pressure homogenization process, encapsulation process or solid dispersion processes.

### DESCRIPTION OF THE INVENTION

Disclosed herein are stable pharmaceutical formulations comprising as active compound Suvorexant, or its salts or its metabolites or derivatives thereof; and at least one primary pharmaceutical excipient.

In an embodiment, said pharmaceutical formulations further comprises at least one secondary pharmaceutical excipient.

We have found that only the selected combinations of primary pharmaceutical excipients and secondary pharmaceutical excipients disclosed in the present invention result in a stable pharmaceutical formulations having improved physicochemical characteristics and enhanced biological performance.

The primary pharmaceutical excipients themselves or together with the secondary pharmaceutical excipients have the function to form a complex structure with Suvorexant, or its salts or its metabolites or derivatives through non-covalent secondary interactions. The secondary interactions can form through electrostatic interactions such as ionic interactions, H-bonding, dipole-dipole interactions, dipole-induced dipole interactions, London dispersion forces, it-it interactions, and hydrophobic interactions. The primary pharmaceutical excipients and secondary pharmaceutical excipients are selected from the group of pharmaceutically accepted excipients which are able to form such complex structures through non-covalent secondary interactions.

In an embodiment, said primary pharmaceutical excipients is chosen from poloxamers (copolymers of ethylene oxide and propylene oxide blocks), copolymers of vinylpyrrolidone and vinyl acetate copolymer, polyvinylpyrrolidone, polyvinyl caprolactam-polyvinyl acetate-polyethylene glycol graft copolymer, hydroxypropylcellulose, ethylene oxide/propylene oxide tetra functional block copolymer, and d-alpha tocopheryl polyethylene glycol 1000 succinate.

In an embodiment, said primary pharmaceutical excipient is polyvinyl caprolactam-polyvinyl acetate-polyethylene glycol graft copolymer or hydroxypropylcellulose.

In an embodiment, said secondary pharmaceutical excipient is chosen from cetylpyridinium chloride (CPC), poloxamers (copolymers of ethylene oxide and propylene oxide blocks), mannitol, polyglycol mono- and di-esters of 12-hydroxystearic acid, dioctyl sodium sulfosuccinate (DSS), sodium acetate (NaOAc), and sodium lauryl sulfate (SDS).

In an embodiment, said secondary pharmaceutical excipient is dioctyl sodium sulfosuccinate or sodium lauryl sulfate and mannitol.

In some embodiments, the pharmaceutical compositions may additionally include one or more pharmaceutically acceptable excipients, auxiliary materials, carriers, active agents or combinations thereof.

In an embodiment, said pharmaceutical formulations have particle size between 10 nm and 600 nm.

In an embodiment said pharmaceutical formulations have particle size in the range between 10 nm and 400 nm.

In an embodiment, said pharmaceutical formulations are instantaneously redispersible in physiological relevant media.

In an embodiment, said pharmaceutical formulations have increased dissolution rate compared to the commercially available form of Suvorexant (BELSOMRA®).

In an embodiment, said pharmaceutical formulations are stable in solid form and in colloid solution and/or dispersion.

In an embodiment, said pharmaceutical formulations have apparent solubility in water is at least 0.15 mg/mL.

In an embodiment, said pharmaceutical formulations exhibits X-ray amorphous character in the solid form.

In an embodiment, said pharmaceutical formulations have PAMPA permeability of at least 6·10⁻⁶ cm/s when dispersed in FaSSiF or FeSSiF media, which does not decrease in time at least for 2 months.

In an embodiment, the variability of absorption and time to maximal plasma concentration of the pharmaceutical formulation of the present invention is significantly reduced compared to the commercially available form of Suvorexant (BELSOMRA®).

In an embodiment, said pharmaceutical formulations have no observable food effect with respect to immediate absorption and more predictable plasma concentration throughout the night and next morning.

In an embodiment said pharmaceutical formulation containing polyvinyl caprolactam-polyvinyl acetate-polyethylene glycol graft copolymer and sodium lauryl sulfate or its pharmaceutical composition according to the invention has characteristic Raman shifts at 426, 568, 640, 680, 701, 794, 846, 880, 921, 953, 1037, 1087, 1199, 1249, 1335, 1374, 1402, 1448, 1505, 1571, 1591, 1616, 1635, 1736, 2691, 2860 and 2938 cm⁻¹; and ATR peaks at 571, 601, 717, 840, 951, 974, 1031, 1084, 1148, 1196, 1236, 1334, 1371, 1421, 1442, 1478, 1570, 1631, 1732, 2857 and 2926 cm⁻¹.

In an embodiment said pharmaceutical formulation containing hydroxypropylcellulose, sodium lauryl sulfate and mannitol or its pharmaceutical composition according to the invention has characteristic Raman shifts at 474, 639, 845, 876, 887, 924, 953, 1053, 1084, 1112, 1129, 1146, 1250, 1297, 1376, 1404, 1453, 1508, 1572, 1587, 1615, 2728, 2850, 2882, 2937, 2918 and 2963cm and ATR peaks at 592, 626, 716, 837, 892, 931, 1026, 1082, 1220, 1251, 1376, 1453, 1571, 1639, 2920, 2848 and 2964 cm⁻¹.

In an embodiment said pharmaceutical formulations comprise
a) Suvorexant, or its salt or its metabolites or derivatives thereof;
b) polyvinyl caprolactam-polyvinyl acetate-polyethylene glycol graft copolymer or hydroxypropylcellulose as primary pharmaceutical excipient; and
c) dioctyl sodium sulfosuccinate or sodium lauryl sulfate and mannitol as secondary pharmaceutical excipient.

In an embodiment, said pharmaceutical formulations comprise a primary pharmaceutical excipient which is polyvinyl caprolactam-polyvinyl acetate-polyethylene glycol graft copolymer or hydroxypropylcellulose and a secondary pharmaceutical excipient which is dioctyl sodium sulfosuccinate; or sodium lauryl sulfate and mannitol, in a total amount comprising from about 1.0 weight % to about 95.0 weight % based on the total weight of the pharmaceutical formulations.

In an embodiment, said pharmaceutical formulations comprise a primary pharmaceutical excipient which is polyvinyl caprolactam-polyvinyl acetate-polyethylene glycol graft copolymer or hydroxypropylcellulose and a secondary pharmaceutical excipient which is dioctyl sodium sulfosuccinate or sodium lauryl sulfate and mannitol, in a total amount comprising from about 50.0 weight % to about 95.0 weight % based on the total weight of the pharmaceutical formulations.

Further disclosed herein are stable pharmaceutical formulations comprising
a. 5-40% by weight of Suvorexant, or its salt or its metabolite or derivatives thereof;
b. 20-90% by weight of a polyvinyl caprolactam-polyvinyl acetate-polyethylene glycol graft copolymer or hydroxypropylcellulose; and
c. 5-40% by weight of dioctyl sodium sulfosuccinate or sodium lauryl sulfate and mannitol.

Disclosed herein is a process for the preparation of stable pharmaceutical formulations of Suvorexant, or its salt or its metabolite or derivatives said process comprising the step of mixing a pharmaceutical solution of Suvorexant, or its salt or its metabolite or derivatives and at least one primary pharmaceutical excipient with an aqueous solution containing optionally at least one secondary pharmaceutical excipient.

In an embodiment said pharmaceutical formulations are obtained via a mixing process.

In an embodiment said pharmaceutical formulations is obtained via a continuous flow mixing process.

In an embodiment said process is performed in a continuous flow instrument.

In an embodiment said continuous flow instrument is a microfluidic flow instrument.

In an embodiment, said pharmaceutical formulations are not obtained via a milling process, high pressure homogenization process, encapsulation process and solid dispersion processes.

In an embodiment, wherein the pharmaceutically acceptable solvent of said pharmaceutically acceptable solution is chosen from methanol, ethanol, isopropanol, n-propanol, acetone, acetonitrile, tetrahydrofuran or combinations thereof.

In an embodiment, said pharmaceutically acceptable solvent is isopropanol or n-propanol which is mixed with said aqueous solution.

In an embodiment, said pharmaceutically acceptable solution and said aqueous solution are miscible with each other.

In an embodiment, said aqueous solution comprises 0.1 to 99.9% weight of the final solution.

In an embodiment, said aqueous solution comprises 50 to 90% weight of the final solution.

In an embodiment, said aqueous solution comprises 50 to 80% weight of the final solution.

In an embodiment, said aqueous solution comprises 50 to 70% weight of the final solution.

In an embodiment, said aqueous solution comprises 50 to 60% weight of the final solution.

In an embodiment, said aqueous solution comprises 45 to 55% weight of the final solution.

In an embodiment, said aqueous solution comprises 50% weight of the final solution.

In an embodiment, said aqueous solution comprises 35 to 45% weight of the final solution.

In an embodiment, said aqueous solution comprises 25 to 35% weight of the final solution.

In an embodiment, said aqueous solution comprises 15 to 25% weight of the final solution.

In an embodiment, said aqueous solution comprises 5 to 15% weight of the final solution.

In an embodiment, pharmaceutical compositions comprise the pharmaceutical formulations together with pharmaceutically acceptable carriers.

In an embodiment, said pharmaceutical compositions are suitable for oral, pulmonary, rectal, colonic, parenteral, intracisternal, intravaginal, intraperitoneal, ocular, otic, local, buccal, nasal, or topical administration.

In an embodiment, said pharmaceutical compositions are suitable for oral administration.

In an embodiment, said pharmaceutical formulations are for use in the manufacture of a medicament for the treatment of insomnia characterized by difficulties with sleep onset and/or sleep maintenance, for the improvement of daytime sleep in shift workers, for treatment of insomnia related to bipolar disorder, treatment of Suvorexant and trauma related insomnia, treatment of insomnia in Parkinson disease, treatment of sleep pressure in hypertensives with insomnia and for treatment of insomnia in Alzheimer's disease.

In an embodiment, said pharmaceutical formulations are for use in the treatment of insomnia characterized by difficulties with sleep onset and/or sleep maintenance, in the improvement of daytime sleep in shift workers, in the treatment of insomnia related to bipolar disorder, in the treatment of Suvorexant and trauma related insomnia, in the treatment of insomnia in Parkinson disease, in the treatment of sleep pressure in hypertensives with insomnia and in the treatment of insomnia in Alzheimer's disease.

In an embodiment, a method of treatment of insomnia characterized by difficulties with sleep onset and/or sleep maintenance, improvement of daytime sleep in shift workers, treatment of insomnia related to bipolar disorder, treatment of Suvorexant and trauma related insomnia, treatment of insomnia in Parkinson disease, treatment of sleep pressure in hypertensives with insomnia and treatment of insomnia in Alzheimer's disease comprises administration of a therapeutically effective amount of pharmaceutical formulations or pharmaceutical compositions as described herein.

In an embodiment said pharmaceutical formulations comprising Suvorexant or its salt or its metabolite; or a combination of active compounds including Suvorexant; a primary pharmaceutical excipient which is polyvinyl caprolactam-polyvinyl acetate-polyethylene glycol graft copolymer or hydroxypropylcellulose; and dioctyl sodium sulfosuccinate or sodium lauryl sulfate and mannitol as a secondary pharmaceutical excipient characterized in that they possess at least one of the following properties:
a) is instantaneously redispersable in physiological relevant media;
b) is stable in solid form and in colloid solution and/or dispersion;
c) has an apparent solubility in water of at least 0.15 mg/mL;
d) has a PAMPA permeability of at least 6·10⁻⁶ cm/s when dispersed in FaSSIF or FeSSIF biorelevant media, which does not decrease in time at least for 2 month;
e) exhibits no observable food effect with respect to immediate absorption and more predictable plasma concentration throughout the night and next morning.

In an embodiment, said pharmaceutical formulations possesses at least two of the properties described in a)-e).

In an embodiment, said pharmaceutical formulations possesses at least three of the properties described in a)-e).

The novel pharmaceutical formulations of the present invention possesses instantaneous redispersibility, increased apparent solubility and permeability, no observable food effect with respect to immediate absorption and more predictable plasma concentration throughout the night and next morning.

The expression Suvorexant is generally used for Suvorexant, or its salts such as Suvorexant hydrochloride, Suvorexant methanesulfonate, Suvorexant dodecyl sulfate and metabolites of Suvorexant formed through the metabolic pathways of Suvorexant included oxidation, hydroxylation (M8, M9, 10a), bis-hydroxylations (M6a, b and c, M7b and c), dechlorination (M16 and M17). In addition dog hepatocytes included a glucuronide of M10a (M12), a glucuronide of M9 (M11), and an apparent water addition (M20).

In an embodiment, said primary pharmaceutical excipient is chosen from poloxamers (copolymers of ethylene oxide and propylene oxide blocks), copolymers of vinylpyrrolidone and vinyl acetate copolymer, polyvinylpyrrolidone, polyvinyl caprolactam-polyvinyl acetate-polyethylene glycol graft copolymer, ethylene oxide/propylene oxide tetra functional block copolymer, hydroxypropylcellulose, and d-alpha tocopheryl polyethylene glycol 1000 succinate (TPGS).

In an embodiment, said primary pharmaceutical excipient is polyvinyl caprolactam-polyvinyl acetate-polyethylene glycol graft copolymer and said secondary pharmaceutical excipient is dioctyl sodium sulfosuccinate, and said pharmaceutical formulation has characteristic Raman shifts at 426, 568, 640, 680, 701, 794, 846, 880, 921, 953, 1037, 1087, 1199, 1249, 1335, 1374, 1402, 1448, 1505, 1571, 1591, 1616, 1635, 1736, 2691, 2860 and 2938 cm⁻¹; and ATR peaks at 571, 601, 717, 840, 951, 974, 1031, 1084, 1148, 1196, 1236, 1334, 1371, 1421, 1442, 1478, 1570, 1631, 1732, 2857 and 2926 cm⁻¹.

In an embodiment, said primary pharmaceutical excipient is hydroxypropylcellulose and said secondary pharmaceutical excipient is sodium lauryl sulfate and mannitol, and said pharmaceutical formulation has characteristic Raman shifts at 474, 639c, 845, 876, 887, 924, 953, 1053, 1084, 1112, 1129, 1146, 1250, 1297, 1376, 1404, 1453, 1508, 1572, 1587, 1615, 2728, 2850, 2882, 2937, 2918 and 2963 cm⁻¹; and ATR peaks at 592, 626, 716, 837, 892, 931, 1026, 1082, 1220, 1251, 1376, 1453, 1571, 1639, 2920, 2848 and 2964 cm⁻¹.

In some embodiments, the pharmaceutical compositions may additionally include one or more pharmaceutically acceptable excipients, auxiliary materials, carriers, active agents or combinations thereof.

In some embodiments, active agents may include agents useful for the treatment of insomnia characterized by difficulties with sleep onset and/or sleep maintenance, improvement of daytime sleep in shift workers, treatment of insomnia related to bipolar disorder, treatment of Suvorexant and trauma related insomnia, treatment of insomnia in Parkinson disease, treatment of sleep pressure in hypertensives with insomnia and treatment of insomnia in Alzheimer's disease.

Another aspect of the invention is the pharmaceutical formulation of the Suvorexant with primary pharmaceutical excipients and secondary pharmaceutical excipients in which the primary pharmaceutical excipients and secondary pharmaceutical excipients preferably are associated or interacted with the Suvorexant, such as the results of a mixing process or a continuous flow mixing process. In some embodiment, the structure of the pharmaceutical formulations of Suvorexant is different from the core-shell type milled particle, precipitated encapsulated particles, micelles and solid dispersions.

The pharmaceutical formulations and compositions of the invention can be formulated: (a) for administration selected from the group consisting of oral, pulmonary, rectal, colonic, parenteral, intracisternal, intravaginal, intraperitoneal, ocular, otic, local, buccal, nasal, and topical administration; (b) into a dosage form selected from the group consisting of liquid dispersions, gels, aerosols, ointments, creams, lyophilized formulations, tablets, capsules; (c) into a dosage form selected from the group consisting of controlled release formulations, fast melt formulations, delayed release formulations, extended release formulations, pulsatile release formulations, and mixed immediate release and controlled release formulations; or (d) any combination of (a), (b), and (c).

The pharmaceutical formulations can be formulated by adding different types of pharmaceutically acceptable excipients for oral administration in solid, liquid, local (powders, ointments or drops), or topical administration, and the like.

In an embodiment, the dosage form of the invention is a solid dosage form, although any pharmaceutically acceptable dosage form can be utilized.

Solid dosage forms for oral administration include, but are not limited to, capsules, tablets, pills, powders (sachet), and granules. In such solid dosage forms, the pharmaceutical formulation of Suvorexant is admixed with at least one of the following: one or more inert excipients (or carriers): (a) fillers or extenders, such as, lactose, sucrose, glucose, mannitol, sorbitol, dextrose, dextrates, dextrin, erythritol, fructose, isomalt, lactitol, maltitol, maltose, maltodextrin, trehalose, xylitol, starches, microcrystalline cellulose, dicalcium phosphate, calcium carbonate, magnesium carbonate, magnesium oxide; (b) sweetening, flavoring, aromatizing and perfuming agents such as saccharin, saccharin sodium, acesulfame potassium, alitame, aspartame, glycine, inulin, neohesperidin dihydrochalcone, neotame, sodium cyclamate, sucralose, tagatose, thaumatin, citric acid, adipic acid, fumaric acid, leucine, malic acid, menthol, propionic acid, tartaric acid; (c) binders, such as cellulose derivatives, acrylic acid derivatives, alginates, gelatin, polyvinylpyrrolidone, starch derivatives, dextrose, dextrates, dextrin, maltose, maltodextrin; (d) disintegrating agents, such as crospovidon, effervescent compositions, croscarmellose sodium and other cellulose derivatives, sodium starch glycolate and other starch derivatives, alginic acid, certain complex silicates and sodium carbonate; (e) solution retarders, such as acrylates, cellulose derivatives, paraffin; (f) absorption accelerators, such as quaternary ammonium compounds; (g) wetting agents, such as polysorbates, cetyl alcohol and glycerol monostearate; (h) lubricants such as talc, stearic acid and its derivatives, solid polyethylene glycols, sodium lauryl sulfate, glyceryl behenate, medium-chain triglycerides or mixtures thereof. For capsules, tablets, and pills, the dosage forms may also comprise buffering agents.

In an embodiment, the dosage form is chosen from water dispersible granules in sachet, water dispersible tablet.

Advantages of the pharmaceutical formulations of Suvorexant of the invention include, but are not limited to (1) physical and chemical stability, (2) instantaneous redispersibility, (3) stability in colloid solution or dispersion in the therapeutic time window, (4) increased apparent solubility and permeability compared to the conventional Suvorexant formulation, (5) no observable food effect with respect to immediate absorption and more predictable plasma concentration throughout the night and next morning., (6) lower the incidence of next day drowsiness as a result of more predictable plasma concentrations the next morning, (7) good processability.

Beneficial features of the present invention are as follows: the good/instantaneous redispersibility and stability of solid pharmaceutical formulations of Suvorexant in water, biologically relevant media, e.g. physiological saline solution, pH=2.5 HCl solution, FessiF and FassiF media and gastro intestinal fluids and adequate stability in colloid solutions and/or dispersion in the therapeutic time window.

In an embodiment, the pharmaceutical formulations of Suvorexant of the present invention have increased apparent solubility and permeability. In some embodiments, the apparent solubility and permeability of the pharmaceutical formulations of Suvorexant is at least 0.15 mg/mL and 6·10⁻⁶ cm/s, respectively.

The pharmaceutical formulations of the present invention possess instantaneous redispersibility, increased apparent solubility and permeability, no observable food effect with respect to immediate absorption and more predictable plasma concentration throughout the night and next morning.

### BRIEF DESCRIPTION OF THE DRAWINGS

FIG. 1. shows the redispersibility of Suvorexant formulations in ultrapurified water.

FIG. 2. shows the redispersibility, stability and PAMPA permeability of Suvorexant formulations in ultrapurified water.

FIG. 3. shows the redispersibility, stability and PAMPA permeability of Suvorexant formulations in ultrapurified water.

FIG. 4. shows the GI dissolution of Suvorexant formulations and Belsomra® in Fasted state conditions.

FIG. 5. shows the PAMPA permeability of Suvorexant pharmaceutical formulations and BELSOMRA®.

FIG. 6. shows the PAMPA permeability of Suvorexant pharmaceutical formulation containing Soluplus at different time points and storage conditions.

FIG. 7. shows the PAMPA permeability of Suvorexant pharmaceutical formulation containing Klucel EF at different time points and storage conditions.

FIG. 8. shows the ATR spectra of A: Suvorexant crystalline, B: Suvorexant freeze-dried Suvorexant, C: Suvorexant formulation, D: Placebo, E: Soluplus, F: DSS.

FIG. 9. shows the Raman spectra of A: Suvorexant crystalline, B: Suvorexant freeze-dried Suvorexant, C: Suvorexant formulation, D: Placebo, E: Soluplus, F:DSS.

FIG. 10. shows the ATR of A: Suvorexant crystalline, B: Suvorexant freeze-dried Suvorexant, C: Suvorexant formulation, D: Placebo, E: Klucel EF, F:DSS, G: Mannitol.

FIG. 11. shows the Raman of A: Suvorexant crystalline, B: Suvorexant freeze-dried Suvorexant, C: Suvorexant formulation, D: Placebo, E: Klucel EF, F: DSS, G: Mannitol.

FIG. 12. shows the SEM photo of Suvorexant formulation.

FIG. 13. shows the pXRD diffractograms of Suvorexant formulations at different time points.

FIG. 14. shows the pharmacokinetic parameters following the oral administration of Suvorexant pharmaceutical formulations, the crystalline compound and crushed BELSOMRA® tablets to fasted rats at 3 mg/kg. N=3.

FIG. 15. shows the pharmacokinetic parameters following the oral administration of Suvorexant pharmaceutical Formulation, and BELSOMRA® tablets to beagle dogs in the fasted state and following a high fat meal at 10 mg dose. N=4.

### EXAMPLES

Selection of Pharmaceutical Formulation of Suvorexant with Improved Material Properties

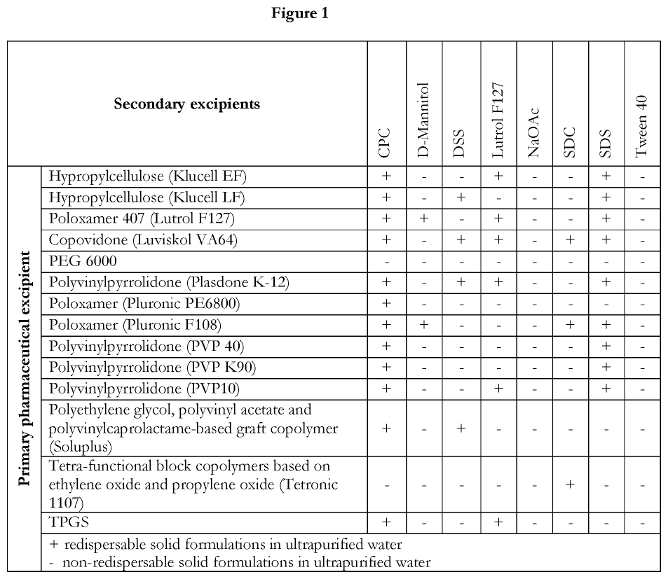

Several primary and secondary pharmaceutical excipients and their combinations were tested in order to select the formulations having instantaneous redispersibility in water as shown in FIG. 1.

In order to find the best pharmaceutical formulation having the optimal composition, the redispersibility, stability of the reconstituted formulations and PAPMPA (parallel artificial membrane permeability assay) permeability of them were investigated. PAMPA permeability measurements were performed as described by M. Kansi et al. (Journal of medicinal chemistry, 41, (1998) pp 1007) with modifications based on S. Bendels et al (Pharmaceutical research, 23 (2006) pp 2525). Permeability was measured in a 96-well plate assay across an artificial membrane composed of dodecane with 20% soy lecithin supported by a PVDF membrane (Millipore, USA). The receiver compartment was phosphate buffered saline (pH=7.0) supplemented with 1% sodium dodecyl sulfate. The assay was performed at room temperature; incubation time was 4 hours in ultrapurified water, FaSSIF and FeSSIF, respectively. The concentration in the receiver compartment was determined by UV-VIS spectrophotometry (Thermo Scientific Multiskan GO microplate spectrophotometer).

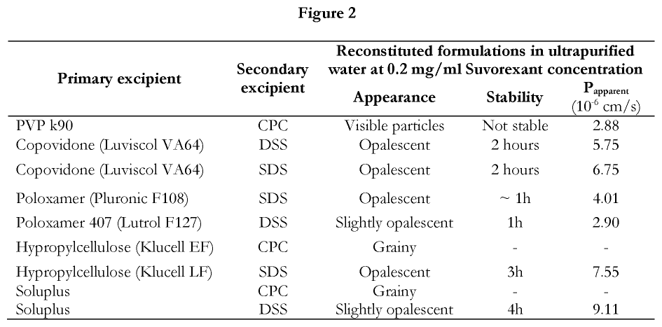

Polyvinyl caprolactam-polyvinyl acetate-polyethylene glycol graft copolymer (Soluplus) and hydroxypropylcellulose (Klucel LF) were selected as primary pharmaceutical excipients; and sodium lauryl sulfate (SDS) and dioctyl sodium sulfosuccinate (DSS) and mannitol were selected as secondary pharmaceutical excipient in order to prepare pharmaceutical formulations of Suvorexant having improved material characteristics (FIG. 2).

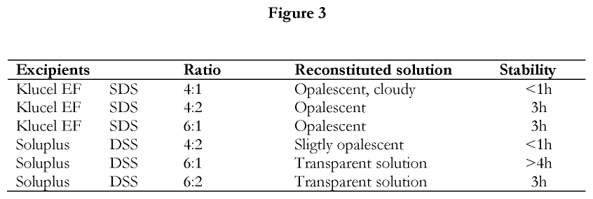

The ratio of the selected primary pharmaceutical excipients and secondary pharmaceutical excipients was optimized. Solid formulations of Suvorexant were prepared by using different ratios of primary pharmaceutical excipients and secondary pharmaceutical excipients. Based on the appearance, the stability and the apparent permeability (PAMPA) of the reconstituted formulations, the optimal ratio of Suvorexant:Soluplus:DSS was found to be 1:6:1; and the optimal ratio of Suvorexant:Klucel EF: SDS: mannitol was found to be 1:4:2: 2(FIG. 3).

### Production of Suvorexant Pharmaceutical Formulations

A colloid solution of Suvorexant pharmaceutical formulation was prepared by mixing 100 mL n-propanol containing 500 mg Suvorexant and 3000 mg polyvinyl caprolactam-polyvinyl acetate-polyethylene glycol graft copolymer and 400 mL water containing 500 mg dioctyl sodium sulfosuccinate in order to produce Suvorexant pharmaceutical formulation. The solution mixture of the Suvorexant pharmaceutical formulation was produced at atmospheric pressure and ambient temperature. The produced solution mixture was frozen on dry-ice and then it was lyophilized using a freeze drier equipped with −110° C. ice condenser, with a vacuum pump. Spray-drying can also be applicable to produce solid powder from the solution mixture of Suvorexant pharmaceutical formulation.

A colloid solution of Suvorexant pharmaceutical formulation was prepared by mixing 100 mL isopropanol containing 500 mg Suvorexant and 400 mL water containing 2000 mg hydroxypropylcellulose, 1000 mg sodium lauryl sulfate and 1000 mg mannitol in order to produce Suvorexant pharmaceutical formulation. The solution mixture of the Suvorexant pharmaceutical formulation was produced at atmospheric pressure and ambient temperature. The produced solution mixture was frozen on dry-ice and then it was lyophilized using a freeze drier equipped with −110° C. ice condenser, with a vacuum pump. Spray-drying can also be applicable to produce solid powder from the solution mixture of Suvorexant pharmaceutical formulation.

The particle size of the reconstituted Suvorexant pharmaceutical formulation was found to be d(50)=300 nm.

### Improved Apparent Solubility of Pharmaceutical Formulations Of Suvorexant

The apparent solubility of the pharmaceutical formulations of Suvorexant was measured by UV-VIS spectroscopy at room temperature. The solid pharmaceutical formulations of Suvorexant were dispersed in ultrapurified water at 0.5 mg/mL Suvorexant concentration. The resulting solutions were filtered by 220 nm disposable syringe filter. The Suvorexant content in the filtrate was measured by UV-VIS spectrophotometer at288 nm and the apparent solubility was calculated. The filtrate may contain particles of Suvorexant pharmaceutical formulation which could not be filtrated out using 220 nm pore size filter.

The apparent solubility of Suvorexant pharmaceutical formulations of the present invention was at least 0.15 mg/mL, when 0.5 mg/mL Suvorexant equivalent formulations were dispersed in ultrapurified water, respectively.

Apparent solubility of Suvorexant pharmaceutical formulation was at least 0.15 mg/mL.

### Improved Dissolution Profile and GI Tract Stability of Suvorexant Pharmaceutical Formulations

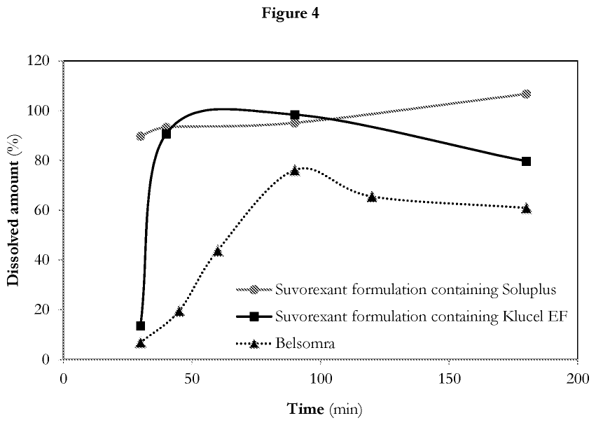

GI simulated dissolution of Suvorexant was measured from the Suvorexant pharmaceutical formulations and BELSOMRA®. The GI dissolution tests were performed by dispersing the Suvorexant pharmaceutical formulation in 20 mL water at 0.5 mg/mL concentration, then it was diluted with 10 mL 1.12 pH SGF solution. After 30 min stirring it was diluted with 10 mL MAB buffer, then with FaSSiF V2 buffer containing the bile salts, resulting in 0.125 mg/mL concentration for the Suvorexant. The solution was then stirred for 3 hours. Samples were taken for HPLC analysis at 30, 40, 90, 180 minutes. The dissolved amount was measured with HPLC after filtration with 0.22 μm pore size filter at time points described above. Dissolution of Suvorexant from the pharmaceutical formulations was instantaneous, within 40 minutes at least 90% of the Suvorexant dissolved from the pharmaceutical formulations. Dissolution from BELSOMRA® was slower compared with the pharmaceutical formulations of the present invention. 76% of Suvorexant dissolved within 90 min from BELSOMRA® and after that precipitation of active ingredient was occurred. (FIG. 4).

### Improved In-Vitro PAMPA Permeability of Suvorexant Pharmaceutical Formulations

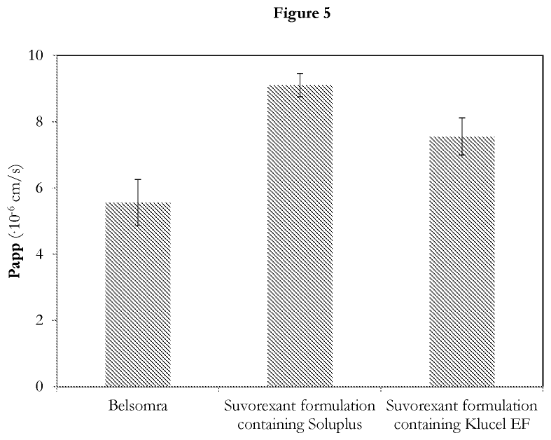

PAMPA permeabilities of Suvorexant pharmaceutical formulations were measured in water and were compared to BELSOMRA®. PAMPA permeability of Suvorexant pharmaceutical formulations was found to be above 7.5·10⁻⁶ cm/s in all tested media, while it was 5.6·10⁻⁶ cm/s for BELSOMRA® (FIG. 5).

### Stability of the Solid Pharmaceutical Formulations of Suvorexant

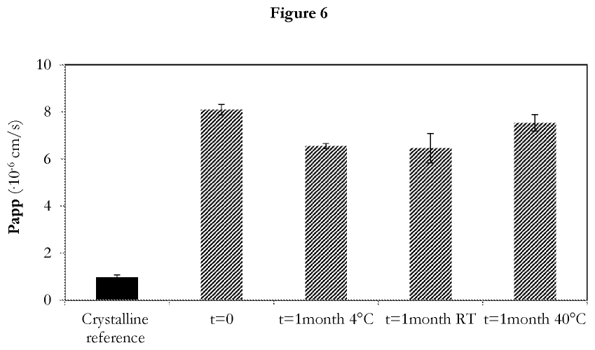

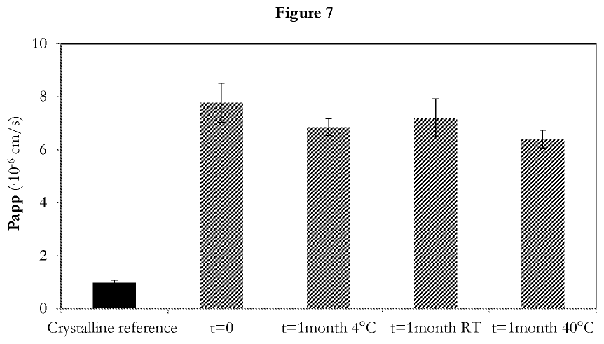

PAMPA permeability of the solid Suvorexant pharmaceutical formulations was used to monitor the physical stability. PAMPA permeability was measured after storage of the solid Suvorexant formulations at different conditions. 1 month storage at RT or 40° C./75% relative humidity showed no significant decrease in the measured PAMPA permeability under any of the conditions tested (FIG. 6 and FIG. 7).

### Structural Analysis

Structural analysis was performed by using HORIB A JobinYvon LabRAM HR UV-VIS-NIR instruments for Raman measurements and Bruker Vertex 70 FT-IR with Bruker Platinum ATR unit, equipped with MCT detector for ATR measurements.

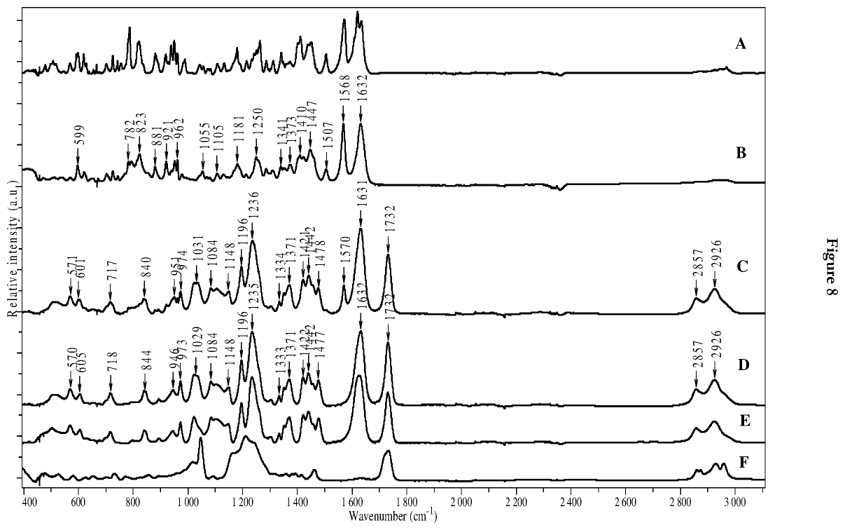

Pharmaceutical formulation of Suvorexant containing polyvinyl caprolactam-polyvinyl acetate-polyethylene glycol graft copolymer (Soluplus) and dioctyl sodium sulfosuccinate (DSS) or its pharmaceutical compositions according to the invention has characteristic ATR peaks at 571, 601, 717, 840, 951, 974, 1031, 1084, 1148, 1196, 1236, 1334, 1371, 1421, 1442, 1478, 1570, 1631, 1732, 2857 and 2926 cm shown in FIG. 8.

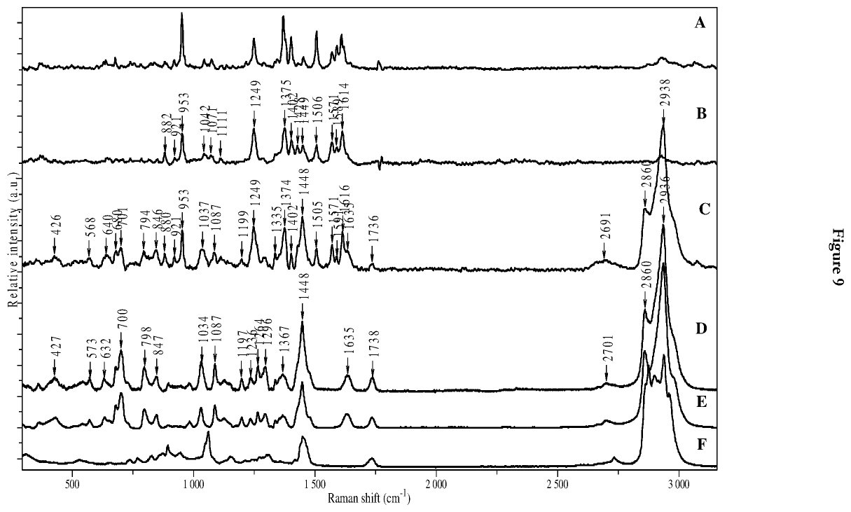

Pharmaceutical formulation of Suvorexant containing polyvinyl caprolactam-polyvinyl acetate-polyethylene glycol graft copolymer (Soluplus) and dioctyl sodium sulfosuccinate or its pharmaceutical compositions according to the invention has characteristic Raman shifts at 426, 568, 640, 680, 701, 794, 846, 880, 921, 953, 1037, 1087, 1199, 1249, 1335, 1374, 1402, 1448, 1505, 1571, 1591, 1616, 1635, 1736, 2691, 2860 and 2938 cm shown in FIG. 9.

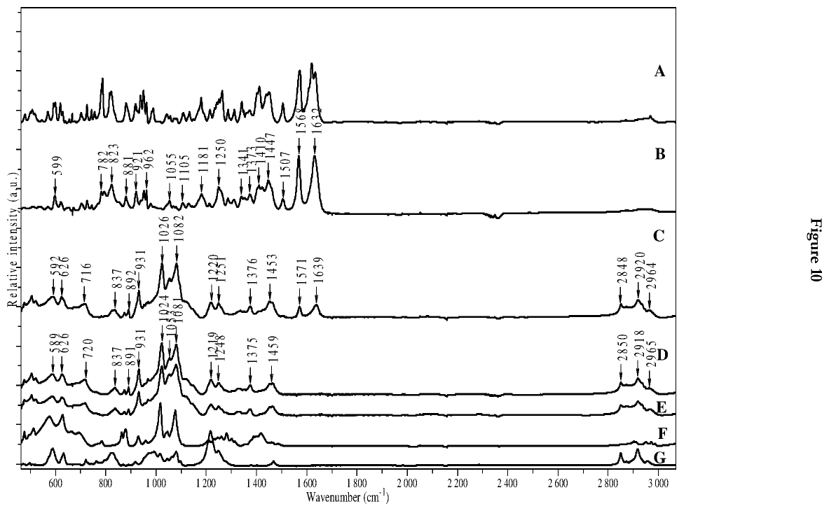

Pharmaceutical formulation of Suvorexant containing hydroxypropylcellulose (Klucel EF), sodium lauryl sulfate (SDS) and mannitol or its pharmaceutical compositions according to the invention has characteristic ATR peaks at 592, 626, 716, 837, 892, 931, 1026, 1082, 1220, 1251, 1376, 1453, 1571, 1639, 2920, 2848 and 2964 cm shown in FIG. 10.

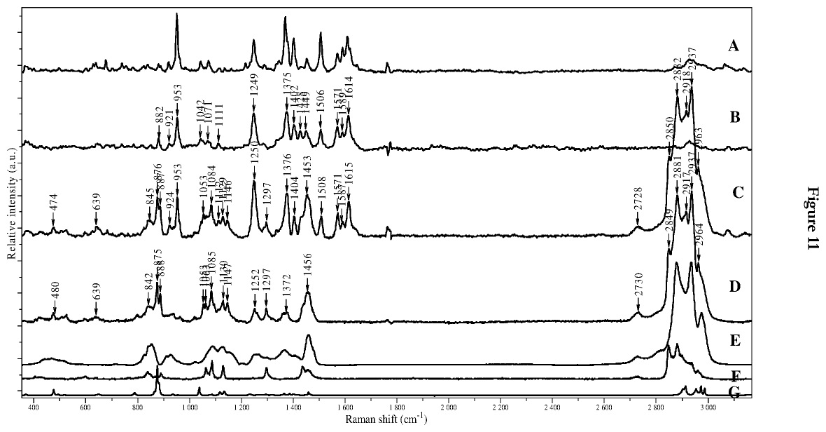

Pharmaceutical formulation of Suvorexant containing hydroxypropylcellulose (Klucel EF), sodium lauryl sulfate (SDS) and mannitol or its pharmaceutical compositions according to the invention has characteristic Raman peaks at 474, 639, 845, 876, 887, 924, 953, 1053, 1084, 1112, 1129, 1146, 1250, 1297, 1376, 1404, 1453, 1508, 1572, 1587, 1615, 2728, 2850, 2882, 2937, 2918 and 2963 cm⁻¹; shown in FIG. 11.

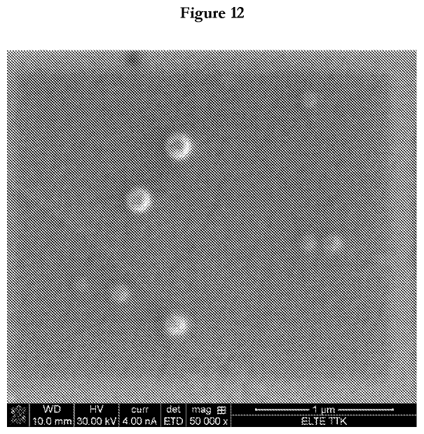

Morphology of Suvorexant formulation was investigated using FEI Quanta 3D scanning electron microscope. Suvorexant formulations of the present invention comprise spherical particles in the size range of less than 200 nm (FIG. 12).

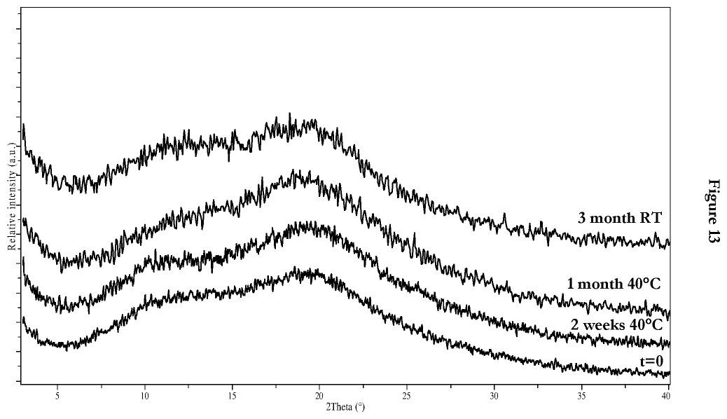

The structure of the Suvorexant pharmaceutical formulations was investigated by powder X-ray diffraction (XRD) analysis (Philips PW1050/1870 RTG powder-diffractometer). The measurements showed that the Suvorexant in the pharmaceutical formulations was XRD amorphous (FIG. 13). The XRD amorphous character of Suvorexant did not change in time for at least 3 months.

### In-Vivo Pharmacokinetics

In-Vivo PK Test

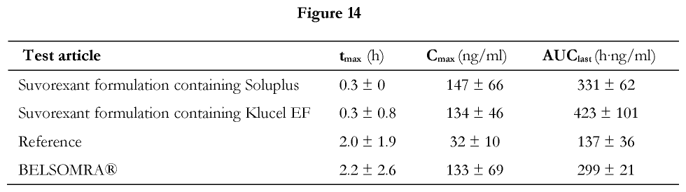

A rat study with oral administration at 3 mg/kg was performed. Two pharmaceutical formulations of the present invention, the crystalline compound and crushed BELSOMRA® tablets were administered and plasma concentrations were measured. Absorption following the administration of the pharmaceutical formulations of the present invention was immediate, while absorption for the crystalline and BELSOMRA® were slower. Formulation 1 of the present invention exhibited ˜1.5-times higher exposure when compared to BELSOMRA® (FIG. 14).

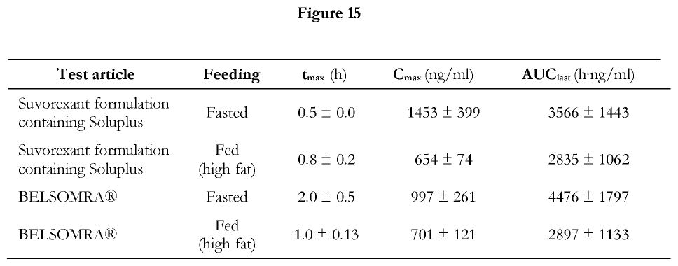

A beagle dog study using the pharmaceutical formulation containing Soluplus of the present invention and BELSOMRA® at a dose of 10 mg/animal was performed in the fasted and fed (high fat) state. Absorption following the administration of the pharmaceutical formulation of the present invention was immediate, while absorption for BELSOMRA® was slower. Food had no significant effect on the very fast absorption profile of the pharmaceutical formulation of the present invention, delay in tₘₐₓ was negligible (FIG. 15).

From the foregoing description, one skilled in the art can easily ascertain the essential characteristics of this invention, and without departing from the spirit and scope thereof, can make various changes and modifications of the invention to adapt it to various usages and conditions.

What is claimed is:

1. Stable pharmaceutical formulations with improved physicochemical characteristics and enhanced biological performance comprising
i. Suvorexant, or its salt, or its metabolite or derivatives thereof;
ii. at least one primary pharmaceutical excipients chosen from polyethylene glycol glycerides composed of from poloxamers (copolymers of ethylene oxide and propylene oxide blocks), copolymers of vinylpyrrolidone and vinyl acetate copolymer, polyvinylpyrrolidone, polyvinyl caprolactam-polyvinyl acetate-polyethylene glycol graft copolymer, ethylene oxide/propylene oxide tetra functional block copolymer, hydroxypropylcellulose and d-alpha tocopheryl polyethylene glycol 1000 succinate; and
iii. optionally, secondary pharmaceutical excipients;
wherein said pharmaceutical formulations have particle size is between 10 nm and 600 nm, and possesses one or more among the following features:
a) is instantaneously redispersible in physiological relevant media;
b) is stable in solid form and in colloid solution and/or dispersion;
c) has apparent solubility in water is of at least 0.15 mg/mL;
d) has PAMPA permeability of at least 6·10⁻⁶ cm/s when dispersed in FaSSiF or FeSSiF media, which does not decrease in time at least for a month;
e) exhibits no observable food effect with respect to immediate absorption and more predictable plasma concentration throughout the night and next morning.

2. The pharmaceutical formulations as recited in claim 1, wherein said pharmaceutical formulations have particle size in the range between 10 nm and 600 nm.

3. The pharmaceutical formulations as recited in claim 2, wherein said pharmaceutical formulations have particle size in the range between 10 nm and 400 nm.

4. The pharmaceutical formulations as recited in claim 1, wherein said pharmaceutical formulations exhibits X-ray amorphous character in the solid form.

5. The pharmaceutical formulations as recited in claim 1, wherein said pharmaceutical formulations possesses at least two of the properties described in a)-e).

6. The pharmaceutical formulations as recited in claim 5, wherein said pharmaceutical formulations possesses at least three of the properties described in a)-e).

7. The pharmaceutical formulations as recited in claim 6, wherein said pharmaceutical formulations possess instantaneous redispersibility, have apparent solubility in water of at least 0.15 mg/mL, exhibits no observable food effect with respect to immediate absorption and more predictable plasma concentration throughout the night and next morning.

8. The pharmaceutical formulations as recited in claim 6, wherein said pharmaceutical formulations possess instantaneous redispersibility, have PAMPA permeability of at least 6·10⁻⁶ cm/s when dispersed in FaSSIF or FeSSIF biorelevant media, which does not decrease in time at least for 1 month, exhibits no observable food effect with respect to immediate absorption and more predictable plasma concentration throughout the night and next morning.

9. The pharmaceutical formulations as recited in claim 5, wherein said pharmaceutical formulations have apparent solubility in water of at least 0.15 mg/mL and PAMPA permeability of at least 6·10⁻⁶ cm/s.

10. The pharmaceutical formulations as recited in claim 6, wherein said pharmaceutical formulations possess instantaneous redispersibility, have apparent solubility in water of at least 0.15 mg/mL, and have PAMPA permeability of at least 6·10⁻⁶ cm/s.

11. The pharmaceutical formulations as recited in claim 1, wherein said primary pharmaceutical excipient is polyvinyl caprolactam-polyvinyl acetate-polyethylene glycol graft copolymer or hydroxypropylcellulose.

12. The pharmaceutical formulations as recited in claim 1, wherein said secondary pharmaceutical excipient is chosen from cetylpyridinium chloride, poloxamers (copolymers of ethylene oxide and propylene oxide blocks), polyglycol mono- and di-esters of 12-hydroxystearic acid, dioctyl sodium sulfosuccinate, sodium acetate, and sodium lauryl sulfate.

13. The pharmaceutical formulations as recited in claim 13, wherein said secondary pharmaceutical excipient is sodium lauryl sulfate, dioctyl sodium sulfosuccinate, and mannitol.

14. The pharmaceutical formulation as recited in claim 1 comprising
a) Suvorexant;
b) polyvinyl caprolactam-polyvinyl acetate-polyethylene glycol graft copolymer as primary pharmaceutical excipient; and
c) dioctyl sodium sulfosuccinate as secondary pharmaceutical excipient;
wherein said pharmaceutical formulation has characteristic Raman shifts at 426, 568, 640, 680, 701, 794, 846, 880, 921, 953, 1037, 1087, 1199, 1249, 1335, 1374, 1402, 1448, 1505, 1571, 1591, 1616, 1635, 1736, 2691, 2860 and 2938; and ATR peaks at 571, 601, 717, 840, 951, 974, 1031, 1084, 1148, 1196, 1236, 1334, 1371, 1421, 1442, 1478, 1570, 1631, 1732, 2857 and 2926 cm⁻¹.

15. The pharmaceutical formulation as recited in claim 1 comprising
a) Suvorexant;
b) hydroxypropylcellulose as primary pharmaceutical excipient and
c) sodium lauryl sulfate and mannitol as secondary pharmaceutical excipients;
wherein said pharmaceutical formulation has characteristic Raman shifts at 474, 639, 845, 876, 887, 924, 953, 1053, 1084, 1112, 1129, 1146, 1250, 1297, 1376, 1404, 1453, 1508, 1572, 1587, 1615, 2728, 2850, 2882, 2937, 2918 and 2963 cm⁻¹; and ATR peaks at 592, 626, 716, 837, 892, 931, 1026, 1082, 1220, 1251, 1376, 1453, 1571, 1639, 2920, 2848 and 2964 cm⁻¹.

16. A pharmaceutical formulation according to either of claim 1 or 13 comprising a primary pharmaceutical excipient which is polyvinyl caprolactam-polyvinyl acetate-polyethylene glycol graft copolymer and secondary pharmaceutical excipient which is sodium-lauryl-sulfate, in a total amount ranging from about 1.0 weight % to about 95.0 weight % based on the total weight of the pharmaceutical formulation.

17. A pharmaceutical formulation according to either of claim 1 or 13 comprising a primary pharmaceutical excipient which is polyvinyl caprolactam-polyvinyl acetate-polyethylene glycol graft copolymer and secondary pharmaceutical excipient which is dioctyl sodium sulfosuccinate, in a total amount ranging from about 50.0 weight % to about 95.0 weight % based on the total weight of the pharmaceutical formulation.

18. A pharmaceutical formulation according to either of claim 1 or 13 comprising a primary pharmaceutical excipient which is hydroxypropylcellulose and secondary pharmaceutical excipients which are sodium lauryl sulfate and mannitol, in a total amount ranging from about 1.0 weight % to about 95.0 weight % based on the total weight of the pharmaceutical formulation.

19. A pharmaceutical formulation according to either of claim 1 or 13 comprising a primary pharmaceutical excipient which is hydroxypropylcellulose and secondary pharmaceutical excipients which are sodium lauryl sulfate and mannitol, in a total amount ranging from about 50.0 weight % to about 95.0 weight % based on the total weight of the pharmaceutical formulation.

20. The pharmaceutical formulations as recited in claim 1, wherein said pharmaceutical formulations have increased dissolution rate.

21. A process for the preparation of stable pharmaceutical formulations as recited in claim 1, said process comprising the step of mixing a pharmaceutically acceptable solution of Suvorexant and at least one primary pharmaceutical excipient which is polyvinyl caprolactam-polyvinyl acetate-polyethylene glycol graft copolymer or hydroxypropylcellulose with an aqueous solution containing at least one secondary pharmaceutical excipient which is dioctyl sodium sulfosuccinate or sodium lauryl sulfate and mannitol.

22. The process as recited in claim 21, wherein said process is performed in a continuous flow instrument.

23. The process as recited in claim 22, wherein said continuous flow instrument is a microfluidic flow instrument.

24. The process as recited in claim 21, wherein the pharmaceutically acceptable solvent of said pharmaceutically acceptable solution is chosen from methanol, ethanol, isopropanol, n-propanol, acetone, acetonitrile, tetrahydrofuran or combinations thereof.

25. The process as recited in claim 24, wherein the pharmaceutically acceptable solvent of said pharmaceutically acceptable solution is isopropanol or n-propanol which is mixed with said aqueous solution of Point 18.

26. The process as recited in claim 21, wherein said pharmaceutically acceptable solution is miscible with said aqueous solution and the aqueous solution comprises 0.1 to 99.9% weight of the final solution.

27. A pharmaceutical composition comprising the stable pharmaceutical formulation as recited in claim 1 together with a pharmaceutically acceptable carriers.

28. The pharmaceutical composition as recited in claim 27, wherein said pharmaceutical composition is suitable for oral, pulmonary, rectal, colonic, parenteral, intracisternal, intravaginal, intraperitoneal, ocular, otic, local, buccal, nasal, or topical administration.

29. The pharmaceutical composition as recited in claim 28, wherein said composition is suitable for oral administration.

30. Pharmaceutical formulations according to claim 1 for use in the treatment of insomnia characterized by difficulties with sleep onset and/or sleep maintenance, in improving daytime sleep in shift workers, in the treatment of insomnia related to bipolar disorder, in the treatment of Suvorexant and trauma related insomnia, in the treatment of insomnia in Parkinson disease, in the treatment of sleep pressure in hypertensives with insomnia and in the treatment of insomnia in Alzheimer's disease.

31. A method of treatment of insomnia characterized by difficulties with sleep onset and/or sleep maintenance, improving daytime sleep in shift workers, treatment of insomnia related to bipolar disorder, treatment of Suvorexant and trauma related insomnia, treatment of insomnia in Parkinson disease, treatment of sleep pressure in hypertensives with insomnia and treatment of insomnia in Alzheimer's disease comprising administration of a therapeutically effective amount of the pharmaceutical formulation according to Point 1 or the pharmaceutical composition according to claim 29.

32. Stable pharmaceutical formulations comprising
a) 5-40% by weight of Suvorexant, or its salt or its metabolites or derivatives thereof;
b) 20-90% by weight of a polyvinyl caprolactam-polyvinyl acetate-polyethylene glycol graft copolymer or hydroxypropylcellulose;
c) 5-40% by weight of dioctyl sodium sulfosuccinate or sodium lauryl sulfate and mannitol
wherein said pharmaceutical formulations have controlled particle size in the range between 10 nm and 600 nm; and
wherein said pharmaceutical formulations are not obtained via a milling process, high pressure homogenization process, encapsulation process or solid dispersion processes.
